(19) United States
(12) Patent Application Publication
(10) Pub. No.: US 2018/0168134 A1
(43) Pub. Date: Jun. 21, 2018

(54) TRANSGENIC MOUSE EXPRESSING INACTIVATED HUMAN IDURONATE-2-SULPHATASE AND METHOD FOR IMPROVING A HUNTER SYNDROME TREATING AGENT USING SAME

(71) Applicant: MOGAM INSTITUTE FOR BIOMEDICAL RESEARCH, Yongin-si (KR)

(72) Inventors: Chihwa KIM, Yongin-si (KR); Jaehyeon LEE, Yongin-si (KR); Eui-Cheol JO, Yongin-si (KR); Jung-Seob KIM, Yongin-si (KR)

(73) Assignee: MOGAM INSTITUTE FOR BIOMEDICAL RESEARCH, Yongin-si (KR)

(21) Appl. No.: 15/848,149

(22) Filed: Dec. 20, 2017

(30) Foreign Application Priority Data
Dec. 20, 2016 (KR) 10-2016-0174514

(51) Int. Cl. A01K 67/027; C12N 9/16; C12N 15/85; G01N 33/68

(52) U.S. Cl. CPC A01K 67/0275; C12N 9/16; C12Y 301/06013; A01K 2267/0306; G01N 33/6854; C12N 2015/8536; A01K 2227/105; C12N 15/8509

(57) ABSTRACT

A transgenic mouse expressing inactivated human iduronate-2-sulphatase (IDS) and a method for improving agents for treating Hunter syndrome using the transgenic mouse are provided. The transgenic mouse expressing the inactivated human IDS, which has immune tolerance to IDS, does not cause an immune response to IDS which is an active component of the agents for treating Hunter syndrome, and thus the transgenic mouse may be effectively used to identify immunogenic factors other than the immunogenicity of the protein components of the agents.

### CROSS REFERENCE TO RELATED APPLICATIONS

This application claims priority based on Korean Application No. 10-2016-0174514, filed Dec. 20, 2016, the content of all of which is incorporated herein by reference in its entirety.

### TECHNICAL FIELD

The present invention relates to a transgenic mouse expressing inactivated human iduronate-2-sulphatase and a method for improving an agent for treating Hunter syndrome treating agent using the transgenic mouse. Specifically, the present invention relates to a transgenic mouse expressing inactivated human iduronate-2-sulphatase in which the cysteine at amino acid position 84 is substituted with threonine, and a method for improving a Hunter syndrome treating agent using the transgenic mouse.

### BACKGROUND ART

Mucopolysaccharidosis is a hereditary metabolic disease caused by defects in enzymes present in lysosomes. Lysosomes are intracellular organs involved in various kinds of degradation processes, which include enzymes involved in electron transfer, oxidative phosphorylation, and metabolism of pyruvic acid, fatty acids and several amino acids, and various hydrolases. In particular, lysosomes are associated with degradation of mucopolysaccharides, mucolipids, and sphingolipids. In addition, many enzymatic defects and gene abnormalities have been reported in the metabolic processes relating to lysosomes and mucopolysaccharidosis.

Specifically, mucopolysaccharidosis clinically manifests various symptoms in the skeletal system, circulatory system, mental function, etc. and may be divided into seven types on the clinical, genetic and biochemical bases. Mucopolysaccharidosis is caused by accumulation of sulfated polysaccharides, such as dermatan sulfate, heparan sulfate, or keratan sulfate, in an organ due to abnormalities of enzymes involved in the metabolism of polysaccharides.

Mucopolysaccharidosis type II, also called Hunter syndrome, is caused by defects of iduronate-2-sulphatase, and Hunter syndrome is divided into two subtypes: a severe form which shows severe symptoms from childhood and a mild form which does not show mental retardation, etc. Specifically, the severe type shows symptoms of mental retardation and physical abnormalities while most of the patients with the severe form die before the age of 15 and those with a mild form shows symptoms such as dwarf, coarse facial features, hepatosplenomegaly, etc. and survive long. Hunter syndrome is a relatively common disease in Korea and shows an X-linked recessive mode of inheritance.

Examples of the agents used for the treatment of Hunter syndrome include ELAPRASE® of Shire Pharmaceuticals Group and HUNTERASE® of Green Cross Corporation. In case of ELAPRASE®, in nonclinical and clinical trials, the positive rate of iduronate-2-sulphatase (hereinafter, IDS)-specific antibody (IgG type) after the administration thereof was reported to be about 50%, whereas, in case of HUNTERASE®, no significant positive rate of anti-drug antibody (hereinafter, ADA) was shown in nonclinical and clinical trials.

Although HUNTERASE® and ELAPRASE® are regarded as similar drugs, these two drugs show a difference with respect to their immunogenicity, and thus it is necessary to identify the causes of ADA production by HUNTERASE® to provide more optimized drugs in the future.

### PRIOR ART DOCUMENT

(Non-Patent Document 1) Chihwa Kim et al, Journal of Human Genetics, 8:1-8, 2016

### DISCLOSURE OF INVENTION

### Technical Problem

In this regard, in order to identify ADA production by immunogenic factors such as aggregation, purity and shape, other than the immunogenicity of the protein itself, of IDS which is an active component of the agents for treating Hunter syndrome, the present inventors prepared a transgenic mouse with immune tolerance to IDS, which expresses an inactivated human IDS having an immunological environment similar to that of Hunter syndrome patients in a state being immunologically tolerant thereto by substituting the cysteine at amino acid position 84 with threonine. As a result, the present inventors confirmed that ADA was not produced in the transgenic mouse with immune tolerance to IDS after IDS was administered thereto.

### Solution to Problem

The present invention provides a targeting vector which comprises a nucleotide sequence of the inactivated human iduronate-2-sulphatase (IDS) in which the cysteine at amino acid position 84 is substituted with threonine.

Additionally, the present invention provides a transgenic zygote obtained by delivering the targeting vector to a zygote of a mouse.

Additionally, the present invention provides a transgenic mouse expressing inactivated human IDS in which the cysteine at amino acid position 84 is substituted with threonine.

Additionally, the present invention provides a transgenic mouse with immune tolerance to IDS produced by mating the transgenic mouse with an IDS heterozygous mouse, in which the IDS protein moiety of the mouse is removed.

Additionally, the present invention provides a method for providing information on the immunogenic factors of a Hunter syndrome treating agent, which includes: i) injecting two or more agents for treating Hunter syndrome to the transgenic mouse with immune tolerance to IDS; ii) confirming whether or not an anti-drug antibody (ADA) is produced in the transgenic mouse with immune tolerance to IDS; and iii) comparing the results of step ii) above obtained by two or more agents for treating Hunter syndrome with each other.

### Advantageous Effects of Invention

The transgenic mouse expressing inactivated human iduronate-2-sulphatase (IDS) of the present invention, which has immune tolerance to IDS, does not cause an immune response to IDS, which is an active component of agents for treating Hunter syndrome, and thus the transgenic mouse may be effectively used to identify immunogenic factors other than the immunogenicity of the protein components of the agents.

### BRIEF DESCRIPTION OF DRAWINGS

FIG. 1 shows a schematic diagram illustrating the targeting vector (pcDNA3.1 (+)-IDS^{C84T}) for preparing an inactivated human IDS transgenic mouse in which the cysteine at amino acid position 84 is substituted with threonine.

FIG. 2 shows an image illustrating the electrophoresis results of a targeting vector after the treatment with NheI and XhoI.

FIG. 3 shows a graph illustrating the quantification of the amount of the inactivated human IDS protein produced by culturing the 293FT cells in which a targeting vector was introduced.

FIG. 4 shows an image of the inactivated human IDS protein produced by culturing the 293FT cells, into which a targeting vector was introduced, and confirmed by western blot.

FIGS. 5a to 5e show images illustrating the results of genomic DNA of mice (10 mice secured out of 96 mice, NO: no template, N/C: normal mouse genomic DNA, P/C: positive control (plasmid DNA)) for the screening of the transgenic mice (IDS^{C84T}) expressing the inactivated human IDS, confirmed by PCR amplification.

FIG. 6 shows images illustrating the results of genomic DNA of mice (selection of #1, #2, #3 and #8 mice having both characteristics of a transgenic mouse and an IDS KO mouse among male mice KO: knock-out, WT: wild-type, Tg: transgenic) for the screening of the transgenic mouse, (IDS^{-/-C84T}), which has immune tolerance to IDS, where only the inactivated human IDS is expressed and the IDS protein of the mice is removed, confirmed by PCR amplification.

FIG. 7 shows a schematic diagram illustrating the IDS drug administration time-point and the sample collection time-point in an immunogenicity measurement experiment using transgenic mice (IDS^{-/-C84T}) which have immune tolerance to IDS.

FIGS. 8a and 8b show images confirming the ADA production through ELISA using the plasma samples collected from the IDS KO mice (IDS^{-/-}) at the 20^{th} week after IDS drug administration and the transgenic mice (IDS^{-/-C84T}) which have immune tolerance to IDS.

FIG. 9 shows a graph illustrating the level of ADA production in the IDS KO mice (IDS^{-/-}) and the transgenic mice (IDS^{-/-C84T}), which have immune tolerance to IDS, up to the 28^{th} week after IDS drug administration.

FIG. 10 shows a schematic diagram illustrating the time-point for the administration of an adjuvant-containing IDS drug and the time-point for sample collection in an immunogenicity measurement experiment using transgenic mice (IDS^{-/-C84T}) which have immune tolerance to IDS.

FIGS. 11a and 11b show images confirming the ADA production through ELISA with respect to the samples of day 48 in the IDS KO mice (IDS^{-/-}) and the transgenic mice (IDS^{-/-C84T}) which have immune tolerance to IDS, after the administration of two kinds of adjuvant-containing IDS drugs.

FIG. 12 shows a graph illustrating the level of ADA production in the IDS KO mice (IDS^{-/-}) and the transgenic mice (IDS^{-/-C84T}), which have immune tolerance to IDS, up to day 54 after the administration of two kinds of adjuvant-containing IDS drugs.

### BEST MODE FOR CARRYING OUT THE INVENTION

The present invention provides a targeting vector, which includes a nucleotide sequence encoding inactivated human iduronate-2-sulphatase (IDS) in which the cysteine at amino acid position 84 is substituted with threonine.

The human IDS is an enzyme involved in the degradation of heparan sulfate and dermatan sulfate and it may cause Hunter syndrome or type II mucopolysaccharidosis when deficient. The human IDS may have the amino acid sequence of SEQ ID NO: 1. In addition, the human IDS may be encoded by the nucleotide sequence of SEQ ID NO: 2.

When a point mutation of converting the cysteine at position 84 to threonine occurs in the human IDS, the formyl glycosylation may not proceed properly and thus the human IDS may not exhibit its activity.

Additionally, the nucleotide sequence included in a targeting vector comprising the nucleotide sequence encoding the inactivated human IDS may be a nucleotide sequence encoding the amino acid sequence of SEQ ID NO: 3. Specifically, the nucleotide sequence encoding the amino acid sequence of SEQ ID NO: 3 may be a nucleotide sequence represented by SEQ ID NO: 4.

The targeting vector has multiple cloning sites (MCS) for cDNA cloning, is capable of replication in host cells, and the vector is not particularly limited as long as the vector has a reporter gene or antibiotic-resistant gene for the selection of the targeting vector. In a specific embodiment of the present invention, the vector used was pcDNA3.1(+) vector, but is not limited thereto.

Additionally, the present invention provides a transgenic zygote obtained by delivering the targeting vector to a zygote of a mouse.

The transgenic zygote may be obtained by injecting the DNA of a targeting vector into zygote of a mouse by microinjection. Specifically, the transgenic zygote may be obtained by inducing superovulation in a female mouse, mating it with a male mouse, confirming the mating, collecting zygotes from the fallopian tube of the female mouse with vaginal plug, loading the target vector DNA solution into a microinjection injection pipette and injecting it into the male pro-nucleus under a microscope.

Additionally, the present invention provides a transgenic (IDS^{C84T}) mouse capable of expressing inactivated human IDS, in which the cysteine at amino acid position 84 is substituted with threonine.

The transgenic mouse may be obtained by implanting a transgenic zygote of a mouse into the uterus of a surrogate mother. It may be confirmed whether the inactivated human IDS gene has been inserted, by extracting the genomic DNA from the tail of the mouse born 19 days after the implantation.

Specifically, the transgenic mouse may be screened by confirming the inactivated human IDS amplified by PCR using primers having the nucleotide sequences of SEQ ID NOS: 5 and 6, which specifically bind to the inactivated human IDS gene.

Additionally, the present invention provides a transgenic (IDS^{-/-C84T}) mouse with immune tolerance to IDS, which is prepared by mating the transgenic (IDS^{C84T}) mouse with the IDS heterozygous (IDS^{-/+}) mouse, and expresses human IDS in which the cysteine at amino acid position 84 is substituted with threonine, and from which the IDS protein moiety is removed.

The IDS heterozygous (IDS^{-/+}) mouse may be obtained by mating a normal mouse with a chimeric mouse which is obtained by introducing a targeting vector in which the IDS gene is deleted into an embryonic stem cell and injecting the transgenic embryonic stem cell into a blastocyst of a zygote.

Additionally, the IDS heterozygous (IDS^{-/+}) mouse may be prepared referring to the technology disclosed in KR Patent No. 10-0884564, but is not limited thereto.

As used herein, the term “transgenic mouse with immune tolerance to IDS” refers to a transgenic mouse, where part of the human IDS gene is substituted, part of the mouse IDS gene is deleted, and which expresses inactivated human IDS but does not express mouse IDS.

Additionally, the present invention provides a method for providing information on the immunogenic factors of a Hunter syndrome treating agent, which includes: i) injecting two or more agents for treating Hunter syndrome to the transgenic mouse with immune tolerance to IDS; ii) confirming whether an anti-drug antibody (ADA) is produced in the transgenic mouse with immune tolerance to IDS; and iii) comparing the results of step ii) above obtained by two or more agents for treating Hunter syndrome with each other.

The ADA may be an antibody to IDS.

The Hunter syndrome treating agent may contain IDS. IDS is an active ingredient of a Hunter syndrome treating agent, and it may be ELAPRASE® or HUNTERASE®. Specifically, the above two agents for treating Hunter syndrome have the same amino acid sequence for the IDS protein, but show a difference with respect to the polymer ratio (HUNTERASE® (1.4%) and ELAPRASE® (3.2%)) and purity (HUNTERASE® (98.9%) and ELAPRASE® (96.9%)) of the IDS protein (Chihwa Kim et al, Journal of Human Genetics, 8:1-8, 2016). The ELAPRASE® and HUNTERASE® as agents for treating Hunter syndrome may be used as a sample for a method for investigating conditions more suitable for immunological inducing action.

The IDS may be a protein in which the amino acid at position 84 of the amino acid sequence of SEQ ID NO: 1 is N-glycosylated.

Additionally, the agents for treating Hunter syndrome may contain a pharmaceutically acceptable additive.

### Mode for the Invention

Hereinafter, the present invention is explained in detail by Examples. The following Examples are intended to further illustrate the present invention without limiting its scope.

I. Preparation of IDS^{-/-C84T} Transgenic Mouse

### EXAMPLE 1

### Preparation of pcDNA3.1(+)-IDS^{C84T} Targeting Vector

For the preparation of a targeting vector expressing inactivated human IDS, the nucleotide sequence of SEQ ID NO: 4 in which a cysteine-encoding DNA sequence (i.e., tgc) was substituted with a threonine-encoding DNA sequence (i.e., acc) in the human normal IDS nucleotide sequence of SEQ ID NO: 2, was cloned into the pcDNA3.1(+) vector.

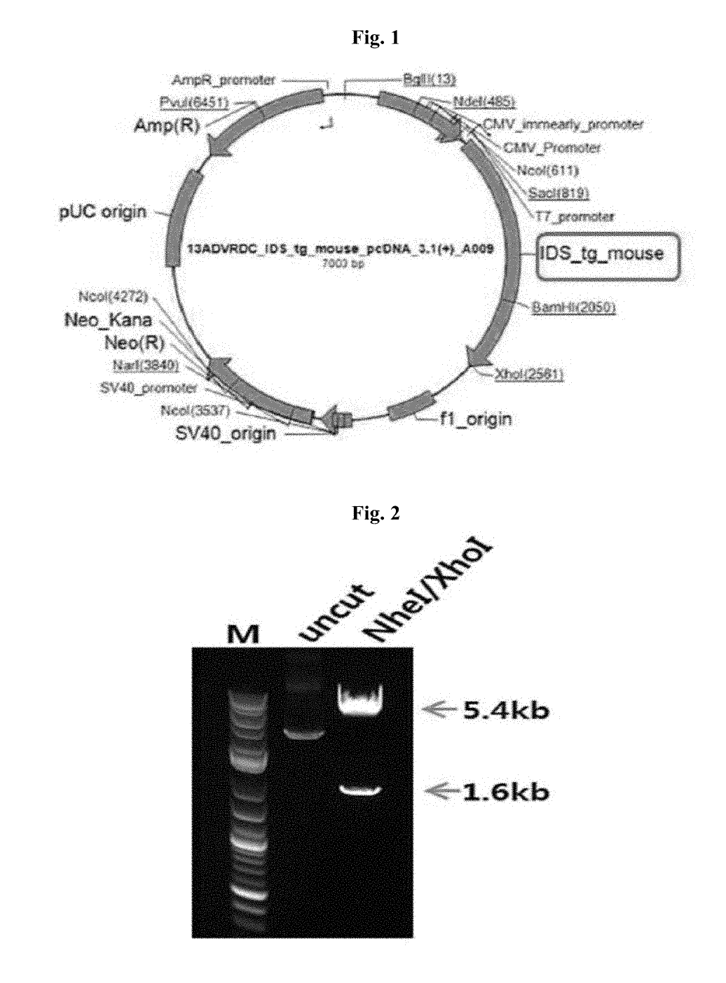

For stable expression of cloned IDS^{C84T} cDNA in mammalian 293FT cells, the pcDNA3.1(+)-IDS^{C84T} vector was prepared by subcloning the cloned IDS^{C84T} cDNA between the Nhel and Xhol restriction enzyme sites of the pcDNA3.1(+) vector (Invitrogen) including the mammalian CMV promoter (FIGS. 1 and 2).

### EXAMPLE 2

### Confirmation of Expression of pcDNA3.1(+)-IDS^{C84T} Targeting Vector

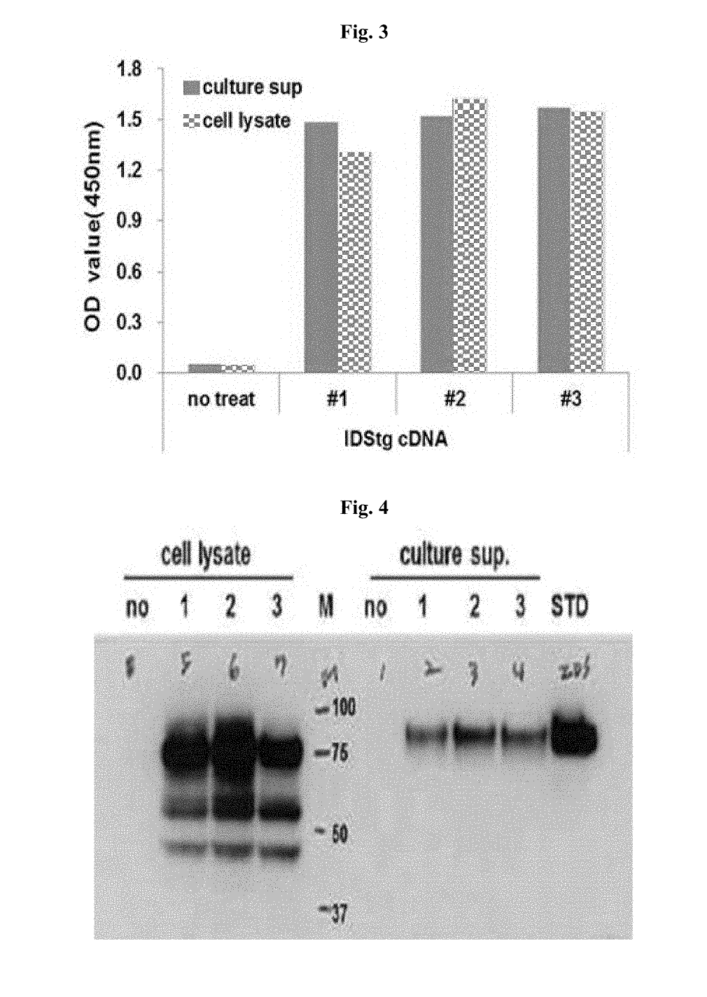

The nucleotide sequence of the pcDNA3.1(+)-IDS^{C84T} vector was analyzed, and as a result, it was confirmed that a point mutation from cysteine at amino acid position 84 to threonine has occurred. Then, the vector was transfected into the 293FT cells, and the cells were cultured and inactivated human IDS was produced. The inactivated human IDS obtained from the cultured 293FT cells were confirmed to be normally expressed, by ELISA and western blotting (FIGS. 3 and 4).

### EXAMPLE 3

### Preparation of Transgenic Zygotes

The pcDNA3.1(+)-IDS^{C84T} targeting vector was degraded with suitable restriction enzymes to prepare linear DNA fragments, electrophoresed on an agarose gel, and the resulting DNA was purified. The purified DNA was prepared to a concentration of about 4 ng/μL.

For the inducement of superovulation, a female mouse was injected with pregnant mare serum gonadotropin (PMSG) and human chorionic gonadotropin (hCG) hormones at 48 hour intervals. After inducing a mating with a male mouse, the female mouse was checked of its vaginal plug the next morning to confirm the result of the mating and zygotes were collected from the fallopian tube with the vaginal plug. Then, the DNA solution was loaded into a microinjection injection pipette and injected into the male pro-nucleus under a microscope.

### EXAMPLE 4

### Preparation of Transgenic (IDS^{C84T} ) Mouse Expressing Inactivated Human IDS

The zygotes into which the targeting DNA prepared in Example 3 was injected was selected and implanted into the surrogate mother's fallopian tube. Transgenic mice were delivered on day 19 after the implantation.

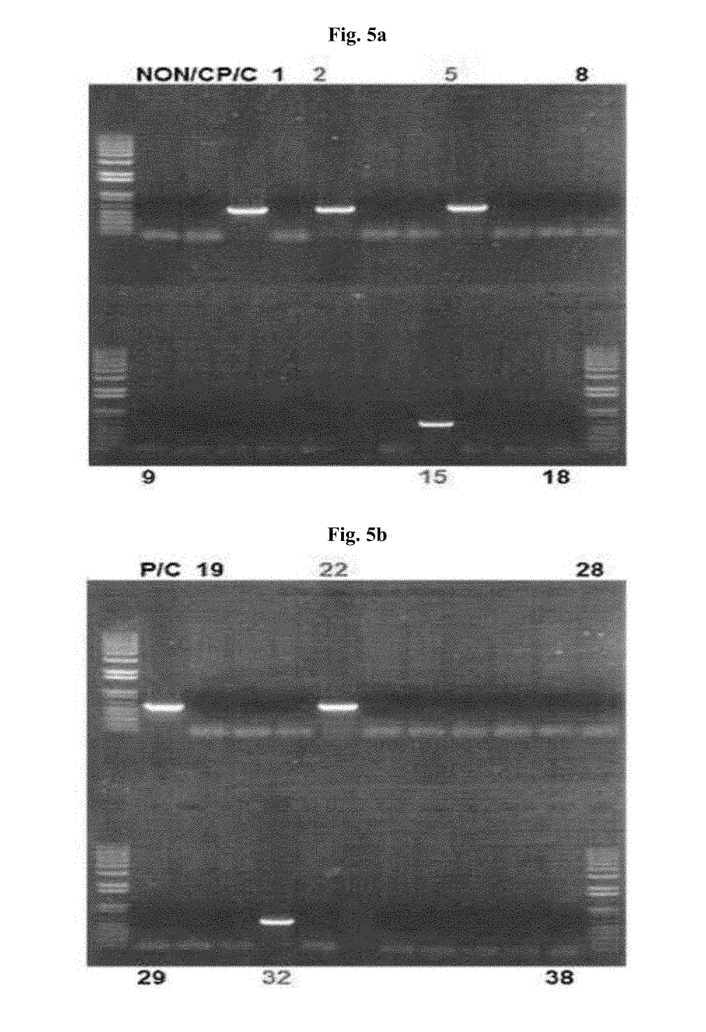

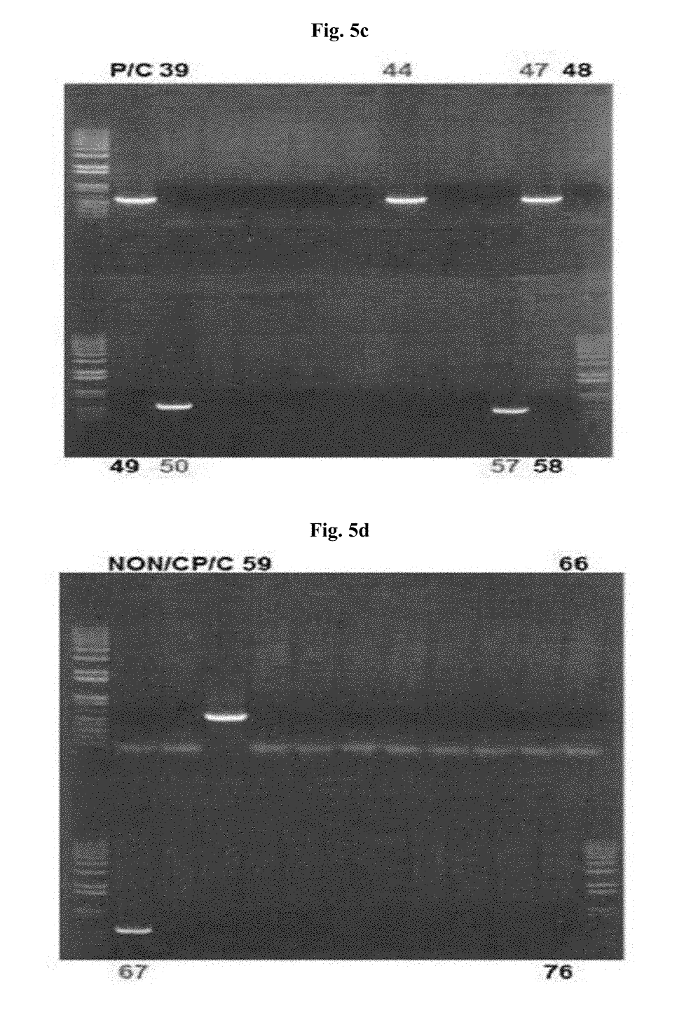

Then, genomic DNA of 4- to 5 week-old mice was extracted from the tails to confirm whether the gene was inserted. PCR was performed using primers having the nucleotide sequences of SEQ ID NOS: 5 and 6, and the transgenic (IDS^{C84T}) mouse expressing the inactivated human IDS were screened and 10 mice were obtained (FIGS. 5A to 5E). The sperm of the transgenic (IDS^{C84T}) mouse was deposited on Nov. 28, 2016, at the Laboratory Animal Resource Center, Korea Research Institute of Bioscience and Biotechnology, as an international depository authority (Deposit No.: AZ00001310).

### EXAMPLE 5

### Screening of Transgenic (IDS^{-/-C84T}) Mouse with Immune Tolerance to IDS

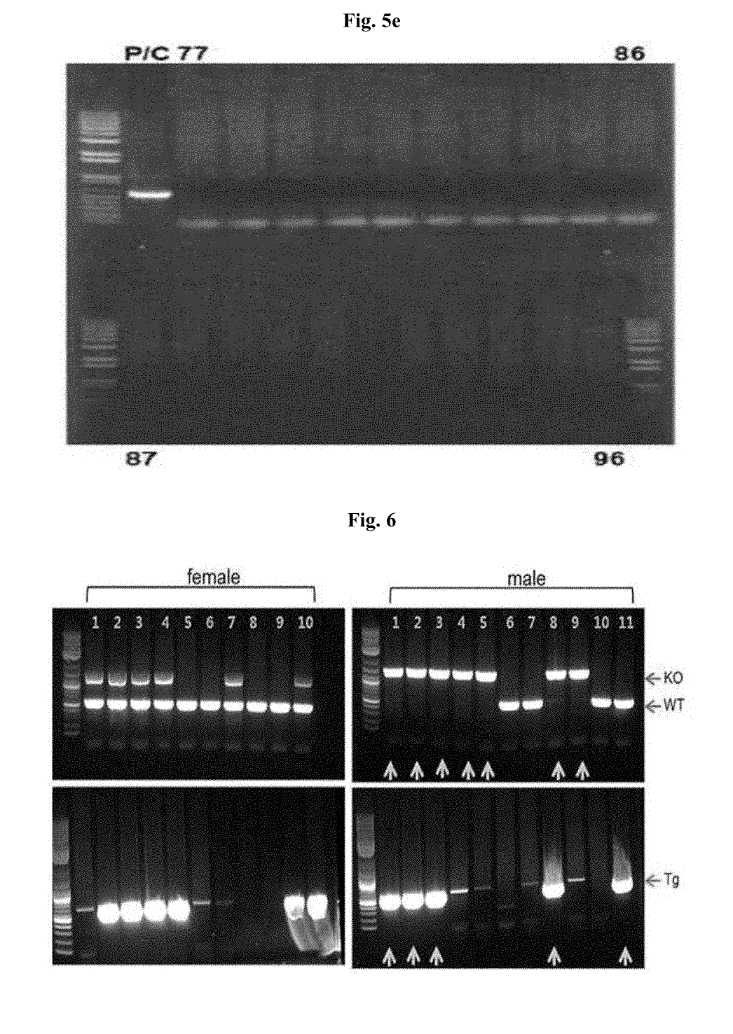

Progenies were obtained by a mating between the transgenic (IDS^{C84T}) mouse and an IDS heterozygous (IDS^{-/+}) mouse. The IDS^{-/-C84T} mouse with immune tolerance to IDS, which has both the IDS deleted gene and the inactivated human IDS gene, was screened by PCR using the tail genomic DNA of 4-5 week old mouse (FIG. 6).

II. Experiment of Measuring Immunogenicity

### EXAMPLE 6

### Confirmation of ADA Production in Accordance with IDS

### EXAMPLE 6-1

### Collection of Samples After IDS Treatment

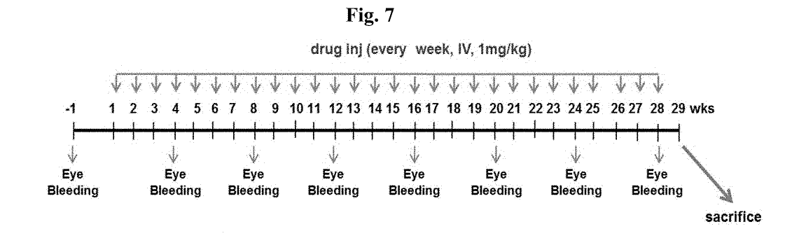

Three kinds of mice (wild type (WT), IDS Knock-out (KO), and transgenic mice with immune tolerance to IDS (IDS^{-/-C84T} )) were divided into 5 groups and drugs were administered at 1 mg/kg once a week for 6 months (Table 1 and FIG. 7). Plasma samples were collected at 3 days before administration and at week 4, 8, 12, 16, 20, 24, and 28 after administration at intervals of 4 weeks. All of the samples were stored at −70° C. until analysis to be analyzed in one batch.

TABLE 1

| | | | Duration of | |
|---|---|---|---|---|
| Group | Drug | Mouse | Administration | Analysis |
| G1 | Saline | WT | Every Week | Collection of |
| | | | for 6 Months | Plasma Sample |
| | | | | (once per month) - |
| | | | | Confirmation of |
| | | | | Formation of Anti- |
| | | | | Drug-Antibody, by |
| | | | | ELISA |
| G2 | Saline | IDS KO | | |
| G3 | IDS 1 mg/kg | | | |
| G4 | Saline | IDS^{-/-C84T} | | |
| G5 | IDS 1 mg/kg | | | |

### EXAMPLE 6-2

### Confirmation of Occurrence of ADA Production After IDS Treatment

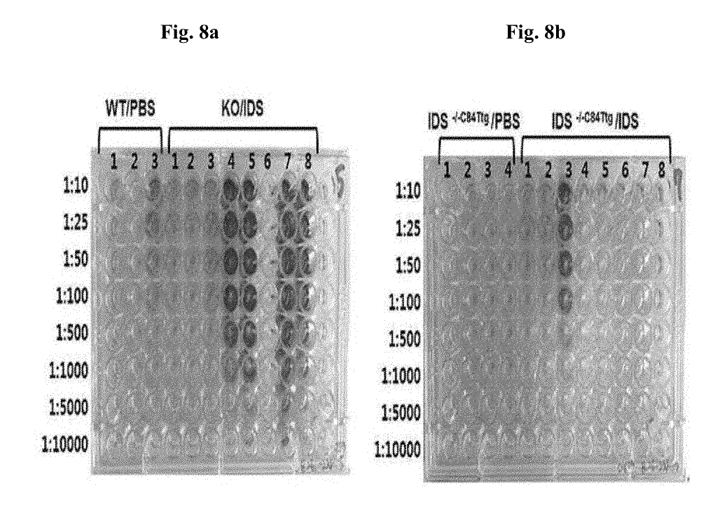

The plasma samples were diluted to 1:10, 1:25, 1:50, 1:100, 1:500, 1:1000, 1:5000 and 1:10,000 with PBS and subjected to ELISA (FIGS. 8a and 8b). Specifically, 10 [symbol] of the IDS protein diluted in PBS was respectively added into a 96-well plate in an amount of 100 [symbol] and placed overnight at 2° C. to 8° C. After discarding the solution in the plate, 300 [symbol] of 1 vol % BSA/PBS was added thereto and allowed to react at room temperature for 1 hour. The diluted plasma samples were injected in an amount of 100 [symbol] into two wells per concentration. The plate was covered with a plate sealer and the samples were allowed to react at room temperature for 2 hours. 300 [symbol] of wash solution (0.1 vol % PBST) was added to each well and washed 3 times. The remaining wash solution was completely removed by hitting against several layers of paper towels.

Anti-mouse-IgG labeled with horseradish peroxidase (HRP) was diluted to 1:2000 with PBS and added to all of the wells in an amount of 100 [symbol] , respectively. The plate was covered with a plate sealer and the samples were allowed to react at room temperature for 1 hour. 300 [symbol] of wash solution (0.1 vol % PBST) was added to each well and washed 3 times. The remaining wash solution was completely removed by hitting against several layers of paper towels.

100 μl of TMB peroxidase substrate solution was added to all of the wells and were allowed to react at room temperature for 15 minutes. 100 μl N sulfuric acid was added to all of the wells to stop the reaction, and the absorbance was measured at a wavelength of 450 nm.

The plasma sample collected at the 20th week was diluted to 1:100 and the occurrence of ADA production was confirmed by ELISA. As a result, the IDS KO mice showed high antibody responses but the transgenic mice with immune tolerance (IDS^{-/-C84T}) did not show any antibody response thereby to confirm that they have immune tolerance to IDS protein (FIGS. 8a and 8b).

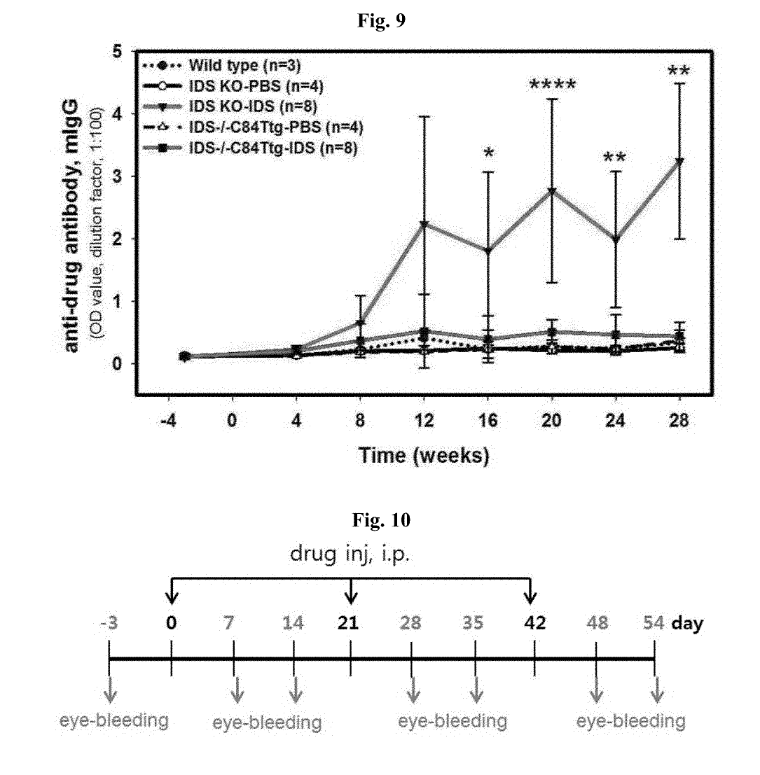

In the case of the IDS KO mice, ADA production was observed from about 8 weeks after administration of the IDS drug, whereas the transgenic mice with immune tolerance (IDS^{-/-C84T}) showed no ADA production by the administration of IDS drug (FIG. 9).

### EXAMPLE 7

### Confirmation of ADA Production in Accordance with IDS Types and Adjuvants

### EXAMPLE 7-1

### Sample Collection After Treatment with Adjuvant-Containing IDS

Three kinds of mice (wild type (WT), IDS Knock-out (KO), and transgenic mice with immune tolerance to IDS (IDS^{-/-C84T})) were divided into 7 groups and intraperitoneally administered with two kinds of adjuvant-containing drugs, HUNTERASE® and ELAPRASE®, and the administration was performed 3 times at intervals of 3 weeks.

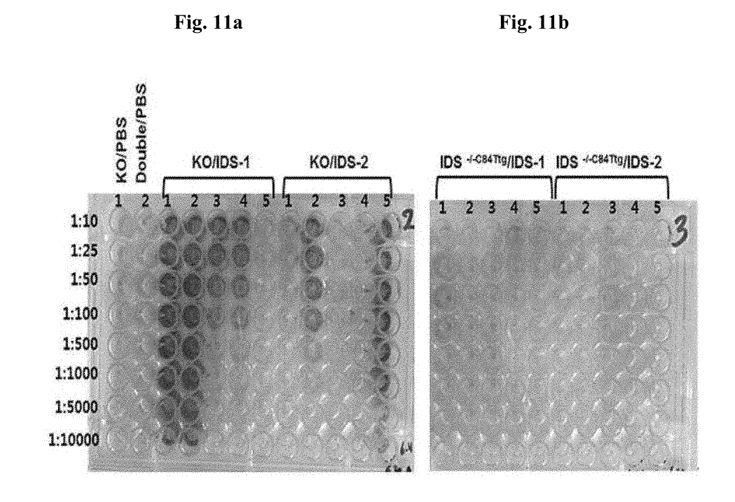

Plasma samples were collected at 3 days before administration, at week 1 (the 7^{th} day) and week 2 (the 14^{th} day) after the first administration (the 0^{th} day), at week 1 (the 28^{th} day) and week 2 (the 35^{th} day) after the second administration (the 21^{st} day), and at week 1 (the 48^{th} day) and week 2 (the 54^{th} day) after the third administration (the 42^{nd} day) (Table 2 and FIG. 10). All of the samples were stored at −70° C. until analysis to be analyzed in one batch.

TABLE 2

| | | | Duration of | |
|---|---|---|---|---|
| Group | Drug | Mouse | Administration | Analysis |
| G1 | PBS | WT | 54 Days | Serum Collection - |
| | | | | Day 3, 7, 14, 28, |
| | | | | 35, 48, and 54 |
| G2 | PBS | IDS KO | | |
| G3 | IDS-1 50 μg | | | |
| | Quil A 5 μg | | | |
| | MPL 5 μg | | | |
| G4 | IDS-2 50 μg | | | |
| | Quil A 5 μg | | | |
| | MPL 5 μg | | | |
| G5 | PBS | IDS^{-/-C84T} | | |
| G6 | IDS-1 50 μg | | | |
| | Quil A 5 μg | | | |
| | MPL 5 μg | | | |
| G7 | IDS-2 50 μg | | | |
| | Quil A 5 μg | | | |
| | MPL 5 μg | | | |

### EXAMPLE 7-2

### Confirmation of Occurrence of ADA Production After Treatment with Adjuvant-Containing IDS

The plasma samples were diluted to 1:10, 1:25, 1:50, 1:100, 1:500, 1:1000, 1:5000, and 1:10,000 with PBS and subjected to ELISA. 10 [symbol] of the IDS protein diluted in PBS was respectively added into a 96-well plate in an amount of 100 [symbol] and was allowed to react overnight at 2° C. to 8° C. After discarding the solution in the plate, 300 [symbol] of 1 vol % BSA/PBS was added thereto and allowed to react at room temperature for 1 hour. The diluted plasma samples were injected in an amount of 100 [symbol] into two wells per concentration. The plate was covered with a plate sealer and the samples were allowed to react at room temperature for 2 hours. 300 [symbol] of wash solution (0.1 vol % PBST) was added to each well and washed 3 times. The remaining wash solution was completely removed by hitting against several layers of paper towels.

Anti-mouse-IgG labeled with horseradish peroxidase (HRP) was diluted to 1:2000 with PBS and added to all of the wells in an amount of 100 [symbol] . The plate was covered with a plate sealer and the samples were allowed to react at room temperature for 1 hour. 300 [symbol] of wash solution (0.1 vol % PBST) was added to each well and washed 3 times. The remaining wash solution was completely removed by hitting against several layers of paper towels.

100 [symbol] of TMB peroxidase substrate solution was added to all of the wells and was allowed to react at room temperature for 15 minutes. 100 [symbol] of 1 N sulfuric acid was added to all of the wells to stop the reaction, and the absorbance was measured at a wavelength of 450 nm.

As a result, it was confirmed that the transgenic mice with immune tolerance (IDS^{-/-C84T}) showed that no ADA was produced by two kinds of adjuvant-containing IDS drugs (FIGS. 11a and 11b).

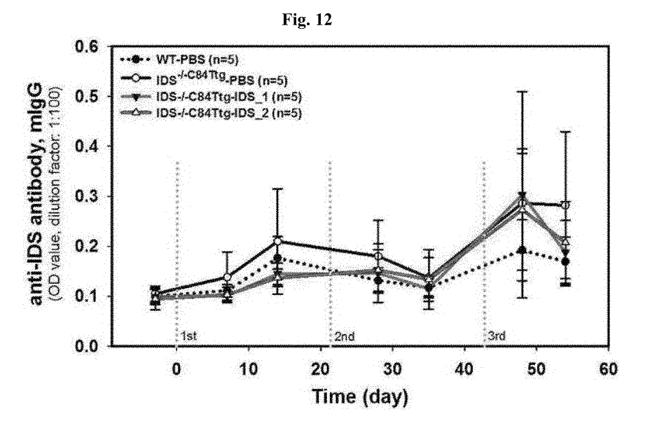

All of the plasma samples collected 7 times were diluted to 1:100 and the occurrence of ADA production was checked by ELISA. It was confirmed that the transgenic mice with immune tolerance (IDS^{-/-C84T}) have immune tolerance to the two kinds of adjuvant-containing IDS drugs (FIG. 12).

1. A targeting vector comprising a nucleotide sequence encoding inactivated human iduronate-2-sulphatase (IDS) in which the cysteine at amino acid position 84 is substituted with threonine.

2. The targeting vector of claim 1, wherein the nucleotide sequence encoding the inactivated human iduronate-2-sulphatase is a nucleotide sequence encoding the amino acid sequence of SEQ ID NO: 3.

3. The targeting vector of claim 1, wherein the nucleotide sequence encoding the inactivated human iduronate-2-sulphatase is a nucleotide sequence encoding the amino acid sequence of SEQ ID NO: 4.

4. A transgenic zygote obtained by delivering the targeting vector of claim 1 to a zygote of a mouse.

5. A transgenic mouse expressing inactivated human iduronate-2-sulphatase in which the cysteine at amino acid position 84 is substituted with threonine.

6. The transgenic mouse of claim 5, wherein the transgenic mouse is obtained by implanting the transgenic zygote of claim 4 into the uterus of a surrogate mother.

7. A transgenic mouse with immune tolerance to iduronate-2-sulphatase (IDS) produced by mating the transgenic mouse of claim 5 with an IDS heterozygous mouse, wherein the IDS protein moiety of the mouse is removed.

8. A method for providing information on the immunogenic factors of a Hunter syndrome treating agent, comprising:
i) injecting two or more agents for treating Hunter syndrome to the transgenic mouse with immune tolerance to IDS of claim 7;
ii) confirming whether or not an anti-drug antibody (ADA) is produced in the transgenic mouse with immune tolerance to IDS; and
iii) comparing the results of step ii) above obtained by two or more agents for treating Hunter syndrome with each other.

9. The method of claim 8, wherein the ADA is an antibody to IDS.

10. The method of claim 8, wherein the agent for treating Hunter syndrome comprises IDS.

11. The method of claim 9, wherein the IDS is an IDS protein represented by the amino acid sequence of SEQ ID NO: 1.

12. The method of claim 8, wherein the agent for treating Hunter syndrome comprises a pharmaceutically acceptable additive.


SEQUENCE LISTING (6 sequences)

SEQ ID NO: 1 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 2 - DNA, 1653 bp, Homo sapiens
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 3 - protein, 550 aa, Artificial Sequence
  amino acid sequence of Human iduronate 2-sulfatase (C84T)
MPPPRTGRGLLWLGLVLSSVCVALGSETQANSTTDALNVLLIIVDDLRPSLGCYGDKLVRSPNIDQLASHSLLFQNAFAQQAVTAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 4 - DNA, 1653 bp, Artificial Sequence
  nucleotide sequence of Human iduronate 2-sulfatase (C84T)
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgaccgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 5 - DNA, 20 bp, Artificial Sequence
  forward primer for iduronate 2-sulfatase (C84T)
cgtgtacggtgggaggtcta

SEQ ID NO: 6 - DNA, 20 bp, Artificial Sequence
  reverse primer for iduronate 2-sulfatase (C84T)
gtgggaaggaccagctgtag